(19) United States
(12) Patent Application Publication
(10) Pub. No.: US 2026/0363534 A1
(43) Pub. Date: Nov. 19, 2026

(54) ENDOSCOPE SYSTEM, CONTROL METHOD, AND CONTROL PROGRAM

(71) Applicant: FUJIFILM Corporation, Tokyo (JP)

(72) Inventors: Masato YOSHIOKA, Kanagawa (JP); Makoto SUGIZAKI, Kanagawa (JP)

(73) Assignee: FUJIFILM Corporation, Tokyo (JP)

(21) Appl. No.: 19/678,162

(22) Filed: May 15, 2026

(30) Foreign Application Priority Data
May 19, 2025 (JP) 2025-083170

(51) Int. Cl. A61B 1/06; A61B 1/00; A61B 1/05

(52) U.S. Cl. CPC A61B 1/0638; A61B 1/00006; A61B 1/05; A61B 1/0655; A61B 1/0684

(57) ABSTRACT

An endoscope system includes a light source device that includes a B light source that outputs B light and a G light source that outputs G light, an endoscope configured to emit illumination light supplied from the light source device to a subject and image the subject to output an image signal, and a control device. The control device is operable in an observation mode in which observation illumination light including at least the B light and the G light is emitted from the endoscope to the subject and a first image generated from the image signal is output, and in a treatment mode in which treatment illumination light including R light output from an R light source is emitted to the subject. The control device controls light amounts of the B light source and the G light source differently between the observation mode and the treatment mode.

### CROSS-REFERENCE TO RELATED APPLICATIONS

This application is based upon and claims the benefit of priority from prior Japanese patent application No. 2025-83170, filed on May 19, 2025, the entire contents of which are incorporated herein by reference.

### BACKGROUND OF THE INVENTION

### 1. Field of the Invention

The disclosed technology relates to an endoscope system, a control method, and a storage medium storing a control program.

### 2. Description of the Related Art

CN112904549A, CN115227188A, CN115227187A, and CN115200703A disclose a light source device of an endoscope. CN115296744C, CN218899384Y, and CN215838929Y disclose a technique related to connection between an endoscope and a control device.

In "OLED catheters for inner-body phototherapy: A case of type 2 diabetes mellitus improved via duodenal photobiomodulation", Jee Hoon Sim, Jinhee Kwon, Hyeonwook Chae, Su-Bon Kim, Hyunsu Cho, Woochan Lee, So Hee Kim, Chun-Won Byun, Sangin Hahn, Do Hyun Park, Seunghyup Yoo, [online], [searched on Apr. 22, 2025], Internet <URL: https://www.science.org/doi/10.1126/sciadv.adh8619>, an internal body treatment method using photobiomodulation (PBM) is disclosed for treatment of type 2 diabetes mellitus (T2DM).

### SUMMARY OF THE INVENTION

A technology of the present disclosure provides an endoscope system, a control method, and a storage medium storing a control program for an endoscope capable of appropriately performing observation and treatment.

An endoscope system according to an aspect of the technology of the present disclosure comprises:
a light source device including a first light source that outputs first light in a first wavelength range;
an endoscope configured to emit illumination light supplied from the light source device to a subject and image the subject to output an image signal; and
a processor,
in which the processor is operable in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject, and
the processor is configured to control a light amount of the first light source differently between the observation mode and the treatment mode.

A control method according to an aspect of the technology of the present disclosure is
a control method of a processor of an endoscope system including a light source device including a first light source that outputs first light in a first wavelength range, an endoscope configured to emit illumination light supplied from the light source device to a subject and image the subject to output an image signal, and the processor, the control method comprising:
operating the processor in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject; and
causing the processor to control a light amount of the first light source differently between the observation mode and the treatment mode.

A non-transitory computer-readable storage medium storing a control program according to an aspect of the technology of the present disclosure is
a non-transitory computer-readable storage medium storing a control program of a processor of an endoscope system including a light source device including a first light source that outputs first light in a first wavelength range, an endoscope configured to emit illumination light supplied from the light source device to a subject and image the subject to output an image signal, and the processor, the control program causing the processor to:
operate in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject; and
control a light amount of the first light source differently between the observation mode and the treatment mode.

According to the technology of the present disclosure, it is possible to appropriately perform observation and treatment.

### BRIEF DESCRIPTION OF THE DRAWINGS

FIG. 1 is an external view showing an endoscope system according to an aspect of the disclosed technology.

FIG. 2 is a block diagram showing a configuration of an endoscope system 2 of FIG. 1.

FIG. 3 is a schematic view showing an example of an appearance of an endoscope connector 18.

FIG. 4 is a schematic view showing an example of an internal configuration of a light source device 59 shown in FIG. 2.

FIG. 5 is a schematic view showing another example of the internal configuration of the light source device 59 shown in FIG. 2.

FIG. 6 is a schematic view showing an example of a hardware configuration for an endoscope 10 and a control device 11 to communicate with each other.

FIG. 7 is a schematic view showing another example of the hardware configuration for the communication between the endoscope 10 and the control device 11.

FIG. 8 is a diagram showing an example of a wavelength spectrum of observation illumination light in an observation mode.

FIG. 9 is a diagram showing an example of a wavelength spectrum of treatment illumination light in a treatment mode.

FIG. 10 is a diagram showing an example of emission ranges of the observation illumination light and the treatment illumination light.

FIG. 11 is a diagram showing an example of an endoscopic image displayed on a monitor 19.

FIG. 12 is a diagram showing an example of a guidance display in an endoscopic image 110.

FIG. 13 is a diagram showing an example of notification of integrated irradiation energy for each division region.

FIG. 14 is a diagram showing an example of switching between the observation illumination light and the treatment illumination light at a predetermined interval.

FIG. 15 is a diagram showing an example of an irradiation instrument for the treatment illumination light, which is different from the light source device 59.

FIG. 16 is a diagram showing an example of a scale 92b of a probe 92 shown in the endoscopic image 110.

FIG. 17 is a diagram showing an example of a holding mechanism that holds a position of a tip part 14 of an insertion part 13 of the endoscope 10 in a lumen of a subject.

FIG. 18 is a diagram showing another example of the wavelength spectrum of the observation illumination light in the observation mode.

FIG. 19 is a diagram showing another example of the wavelength spectrum of the treatment illumination light in the treatment mode.

FIG. 20 is a diagram showing another example of the division regions of the endoscopic image 110.

### DESCRIPTION OF THE PREFERRED EMBODIMENTS

### Endoscope System

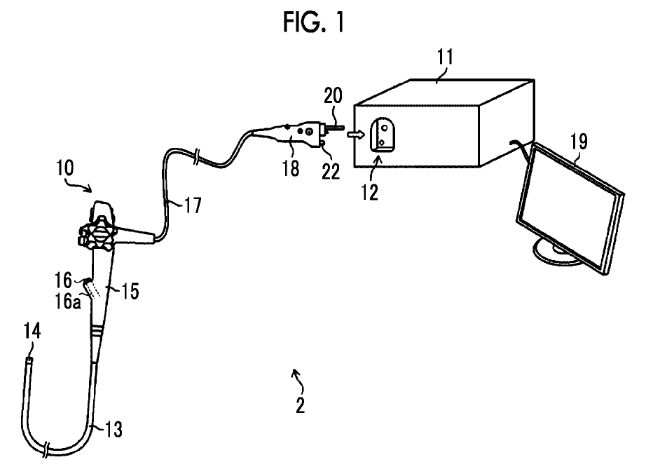

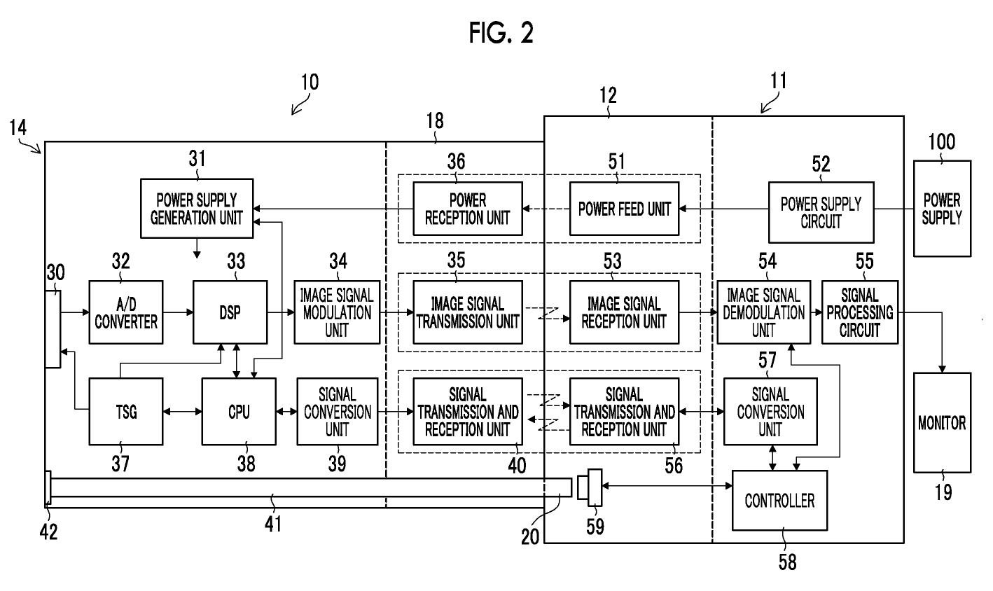

FIG. 1 is an external view showing an endoscope system according to an aspect of the disclosed technology. FIG. 2 is a block diagram showing a configuration of an endoscope system 2 of FIG. 1.

As shown in FIG. 1, the endoscope system 2 is an endoscope apparatus comprising an endoscope 10, a control device 11, and a monitor 19. The control device 11 integrally controls the entire endoscope system 2 in accordance with an operation of an operator input from an input device (an operation switch, a keyboard, a mouse, or the like) (not shown).

The endoscope 10 is an example of a flexible endoscope, and includes a flexible insertion part 13 to be inserted into a body cavity of a patient, an operation unit 15 provided at a proximal end portion of the insertion part 13, a universal cord 17 provided at the operation unit 15, and an endoscope connector 18 provided at an end part of the universal cord 17 and connected to a connector 12 of the control device 11. The endoscope 10 is not limited to a flexible endoscope, and may be another type of endoscope, such as a rigid endoscope.

An observation window, an illumination window (illumination window 23 described later), and the like are provided on a distal end surface of the insertion part 13. A tip part 14 that constitutes a distal end of the insertion part 13 is provided with an imaging unit 30 (see FIG. 2) including an objective optical system that forms an optical image of subject light from a part to be observed taken in through the observation window, and an imaging element that converts the optical image formed by the objective optical system into an image signal. The imaging element is, for example, a charge-coupled device (CCD) image sensor, a complementary metal-oxide-semiconductor (CMOS) image sensor, or the like.

The image signal output from the imaging unit 30 is transmitted to the endoscope connector 18 by a transmission cable inserted into and disposed in the endoscope connector 18 through the inside of the insertion part 13, the operation unit 15, and the universal cord 17.

A light-emitting unit of a light guide 41 that transmits light to be emitted to the part to be observed from the illumination window is disposed in the tip part 14. The light guide 41 is inserted into and disposed in the endoscope connector 18 through the inside of the insertion part 13, the operation unit 15, and the universal cord 17. A light guide rod 20 connected to the light guide 41 is provided to protrude from the endoscope connector 18.

The operation unit 15 includes an angle knob for adjusting the orientation of the distal end surface of the insertion part 13 in vertical and horizontal directions, an air/water supply button for ejecting air and water from the distal end surface of the insertion part 13, a release button for recording a still image of a captured image, and the like. The orientation of the distal end surface of the insertion part 13 is adjusted by bending a bendable part provided in the vicinity of a proximal end side of the tip part 14.

The universal cord 17 is covered with an outer wall part that is tubular and elongated, and has flexibility. The transmission cable and the light guide 41 described above, which are inserted into and disposed in cavity portions inside the insertion part 13 and inside the operation unit 15, the air/water supply tube, and the like are inserted into and disposed in a pipe inside the outer wall part.

The operation unit 15 is provided with a treatment tool insertion port 16 into which an endoscope treatment tool such as forceps can be inserted. In addition, the insertion part 13 is provided with a treatment tool channel 16a that connects the treatment tool insertion port 16 and an opening portion provided in an edge surface of the tip part 14. By inserting the endoscope treatment tool such as forceps into the treatment tool channel 16a from the treatment tool insertion port 16, a distal end of the endoscope treatment tool can be exposed from the opening portion of the distal end surface of the insertion part 13.

The endoscope connector 18 is connected to the connector 12 of the control device 11. The endoscope connector 18 and the connector 12 perform the supply of power from the control device 11 to the endoscope 10, the transmission of the image signal from the endoscope 10 to the control device 11, and the transmission and reception of a control signal between the endoscope 10 and the control device 11 in a noncontact manner without physical connection of electrical wires.

As shown in FIG. 2, the endoscope 10 includes a power reception unit 36 provided in the endoscope connector 18. The control device 11 includes a power feed unit 51 provided in the connector 12. Power required for driving an internal circuit of the endoscope 10 is supplied from the control device 11 by a noncontact power supply means consisting of the power feed unit 51 of the control device 11 and the power reception unit 36 of the endoscope 10.

The noncontact power supply means is a means for transmitting and receiving power in a noncontact manner by using electromagnetic coupling. In a case where the endoscope connector 18 is mounted on the connector 12, the power feed unit 51 and the power reception unit 36 are disposed close to each other at a distance at which electromagnetic coupling is possible, and a state is set in which power transmission from the power feed unit 51 to the power reception unit 36 is possible in a noncontact manner. The power feed unit 51 is connected to a commercial power supply 100 outside the control device 11 via a power supply circuit 52. Power supplied from the commercial power supply 100 and generated by the power supply circuit 52 is supplied to the power feed unit 51. Power is supplied from the power feed unit 51 to the power reception unit 36 in a noncontact manner by the power supplied from the power supply circuit 52 to the power feed unit 51. The power reception unit 36 receives power from the power feed unit 51 in a noncontact manner.

It is preferable that the power feed unit 51 is a primary coil connected to the power supply circuit 52 and that the power reception unit 36 is a secondary coil electromagnetically coupled to the primary coil. Examples of the structure of the primary coil and the secondary coil include a structure having a substrate having a flat surface and a coil spirally wound on the flat surface.

The endoscope 10 includes an image signal transmission unit 35 provided in the endoscope connector 18, a signal transmission and reception unit 40 provided in the endoscope connector 18, a power supply generation unit 31 that generates a power supply voltage to be supplied to each unit of the endoscope 10 from power received by the power reception unit 36, an analog/digital (A/D) converter 32, a digital signal processor (DSP) 33, an image signal modulation unit 34, a timing signal generator (TSG) 37, a central processing unit (CPU) 38 that controls each unit of the endoscope 10, and a signal conversion unit 39.

The image signal output from the imaging unit 30 is changed from an analog signal to a digital signal by the A/D converter 32. The image signal output from the A/D converter 32 is transmitted to the DSP 33. The DSP 33 performs necessary processing such as amplification, gamma correction, and white balance processing on the image signal from the A/D converter 32.

The image signal modulation unit 34 performs optical modulation based on the image signal to generate an image light signal in order to transmit the image signal from the DSP 33 to the control device 11 by optical communication.

The image signal transmission unit 35 irradiates the control device 11 with light (light based on the image signal) in accordance with the image light signal generated by the image signal modulation unit 34. The image signal transmission unit 35 may be a light-emitting device that can emit light for optical communication, and examples thereof include a laser light-emitting element and a light-emitting diode. The laser light-emitting element refers to an element that emits laser light, which is coherent light, and examples thereof include a gas laser, a solid-state laser, and a semiconductor laser.

The signal conversion unit 39 performs optical modulation based on the control signal and demodulation of the control signal in order to transmit and receive the control signal that needs to be transmitted and received between the endoscope 10 and the control device 11 via optical communication. The control signal includes a first control signal transmitted from the control device 11 to the endoscope 10 in order to control the imaging unit 30 and the like, and a second control signal transmitted from the endoscope 10 to the control device 11.

The signal conversion unit 39 performs optical modulation based on the second control signal output from the CPU 38 to generate a second control light signal. The signal conversion unit 39 demodulates the electric signal output from the signal transmission and reception unit 40 to obtain a first control signal.

The signal transmission and reception unit 40 includes a light-emitting device that irradiates the control device 11 with light (light based on the second control signal) in accordance with the second control light signal generated by the signal conversion unit 39, and a light-receiving device that receives light (light based on the first control signal) in accordance with the first control light signal modulated based on the first control signal generated by the control device 11.

Examples of the light-emitting device of the signal transmission and reception unit 40 include a laser light-emitting element and a light-emitting diode. Examples of the light-receiving device of the signal transmission and reception unit 40 include a light-receiving element such as a semiconductor device such as a photodiode or a phototransistor.

The control device 11 includes an image signal reception unit 53 provided in the connector 12, a signal transmission and reception unit 56 provided in the connector 12, an image signal demodulation unit 54, a signal processing circuit 55, a signal conversion unit 57, and a controller 58.

The image signal reception unit 53 is a light-receiving device that receives light emitted from the image signal transmission unit 35 and that converts the light into an electric signal, and examples thereof include a light-receiving element such as a semiconductor device such as a photodiode or a phototransistor.

The image signal demodulation unit 54 demodulates the image signal transmitted from the endoscope 10 based on the electric signal output from the image signal reception unit 53. The image signal is processed by the signal processing circuit 55 and output to the monitor 19. In addition, the image signal is transmitted to the controller 58 and is used to generate a control signal for adjusting the light amount by a so-called auto exposure (AE) function or the like, or is processed to generate an endoscopic image, and the endoscopic image is recorded in a memory or the like.

The signal conversion unit 57 performs optical modulation based on the first control signal output from the controller 58 to generate a first control light signal. The signal conversion unit 57 demodulates the electric signal output from the signal transmission and reception unit 56 to obtain a second control signal.

The signal transmission and reception unit 56 includes a light-emitting device that irradiates the light-receiving device of the signal transmission and reception unit 40 of the endoscope 10 with light (light based on the first control signal) in accordance with the first control light signal generated by the signal conversion unit 57, and a light-receiving device that receives the light emitted from the light-emitting device of the signal transmission and reception unit 40 of the endoscope 10.

Examples of the light-emitting device of the signal transmission and reception unit 56 include a laser light-emitting element and a light-emitting diode. Examples of the light-receiving device of the signal transmission and reception unit 56 include a light-receiving element such as a semiconductor device such as a photodiode or a phototransistor.

In a case where the endoscope connector 18 is mounted on the connector 12 of the control device 11, the image signal transmission unit 35 and the image signal reception unit 53 are disposed close to each other at a distance at which optical communication can be performed, and a state is set in which the image signal transmission unit 35 and the image signal reception unit 53 can perform optical communication in a noncontact manner. In addition, the signal transmission and reception unit 40 and the signal transmission and reception unit 56 are disposed close to each other at a distance at which optical communication can be performed, and a state is set in which noncontact optical communication can be performed between the signal transmission and reception unit 40 and the signal transmission and reception unit 56. As described above, in the present embodiment, the communication of the image signal and the control signal between the endoscope 10 and the control device 11 is performed by the noncontact optical communication means. A method of optical communication is not particularly limited, and various methods can be employed. Wireless communication or magnetic communication can also be used instead of the optical communication.

Although the configuration in which the communication via noncontact is performed between the endoscope connector 18 and the connector 12 has been described, a configuration may be adopted in which the endoscope connector 18 and the connector 12 are connected by an electrical contact and electrical communication is performed between the endoscope connector 18 and the connector 12.

The control device 11 comprises a light source device 59. The light source device 59 has, for example, a light source including a xenon lamp, and a semiconductor device such as a laser diode or a light-emitting diode. In a case where the endoscope connector 18 is mounted on the connector 12 of the control device 11, the light guide rod 20 of the endoscope 10 is connected to the connector 12, and the light-emitting unit of the light source device 59 and the light guide rod 20 are aligned with each other. As a result, the light from the light source device 59 is transmitted to the tip part 14 via the light guide rod 20 and the light guide 41.

The controller 58 is mainly configured with a processor, controls each unit of the control device 11, and transmits a control signal to the CPU 38 and the like constituting an internal circuit of the endoscope 10 to control the entire endoscope system 2. The processor will be described below.

In addition, the endoscope system 2 comprises an illumination window 42 provided at the tip part 14 (tip part) of the insertion part 13 of the endoscope 10. The light output from the light source device 59 is emitted from the illumination window 42. A plurality of illumination windows 42 may be provided.

### Endoscope Connector 18

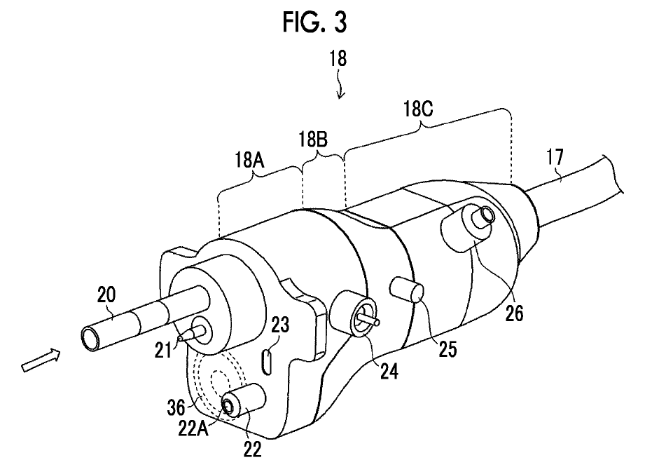

FIG. 3 is a schematic view showing an example of the appearance of the endoscope connector 18. The endoscope connector 18 includes a light guide rod 20 that protrudes from the endoscope connector 18 toward the connector 12 (not shown) and an image signal transmission connector 22.

The endoscope connector 18 can be configured with, for example, a first connector case 18A, a second connector case 18B, and a third connector case 18C in order from a side connected to the connector 12 of the control device 11.

The light guide rod 20 protrudes toward the connector 12 from the first connector case 18A having a connection surface to the connector 12. An air supply mouthpiece 21 is provided below the light guide rod 20 substantially parallel to the light guide rod 20. The air supply mouthpiece 21 communicates with an air and water feeding pipe line provided in the endoscope 10 to supply air and water to the tip part 14 of the endoscope 10.

The image signal transmission connector 22 protrudes from the connection surface with the connector 12 in the first connector case 18A in a direction of insertion toward the connector 12. The image signal transmission connector 22 is used for aligning the image signal transmission unit 35 of the endoscope 10 with the image signal reception unit 53 of the control device 11. In particular, the image signal transmission unit 35 is disposed in an extension direction of a central axis of the image signal transmission connector 22. A window 22A is provided at a distal end of the image signal transmission connector 22 in order to transmit light. Light passes through the window 22A, and transmission and reception of light based on the image signal are performed between the image signal transmission unit 35 and the image signal reception unit 53.

An illumination window 23 is provided at a position corresponding to the signal transmission and reception unit 40 on the connection surface with the connector 12 in the first connector case 18A. Through the illumination window 23, transmission and reception of light based on the control signal are performed between the signal transmission and reception unit 40 and the signal transmission and reception unit 56.

The power reception unit 36 is disposed inside the first connector case 18A and at a position close to the connection surface with the connector 12 in the first connector case 18A. Since the power reception unit 36 is disposed inside the first connector case 18A, it is not exposed to the outside.

An air/water supply connector 24 is provided on a side surface of the first connector case 18A. The air/water supply connector 24 is connected to a water supply tank (not shown). By operating an air/water supply button of the operation unit 15, air or water can be supplied to the tip part 14. The water supplied to the tip part 14 removes the dirt on a lens surface of the tip part 14. In addition, the air supplied to the tip part 14 expands a lumen of the patient or removes water droplets on the lens.

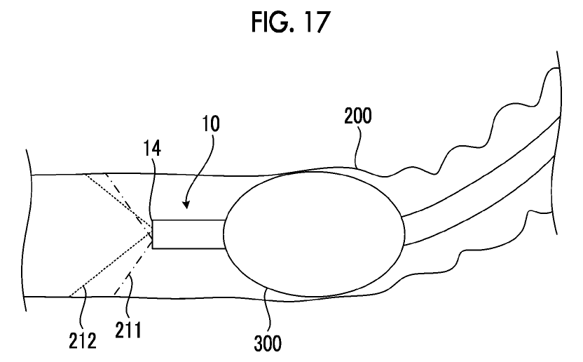

For example, a balloon connector 25 is provided on a side surface of the second connector case 18B. By connecting a tube to the balloon connector 25, a balloon (for example, see FIG. 17) provided in the insertion part 13 can be inflated and contracted. In a case of the endoscope 10 in which the balloon is not provided in the insertion part 13, it is not necessary to provide the balloon connector 25 in the endoscope connector 18.

A ventilation connector 26 is provided on a side surface of the third connector case 18C. The ventilation connector 26 enables a leak test for examining air leakage of the insertion part 13. The ventilation connector 26 communicates with the inside of the endoscope connector 18. Since the inside of the endoscope connector 18 communicates with the inside of each of the universal cord 17, the operation unit 15, and the insertion part 13, the ventilation connector 26 communicates with the inside of the insertion part 13.

The universal cord 17 protrudes from an end part of the third connector case 18C.

### Light Source Device 59

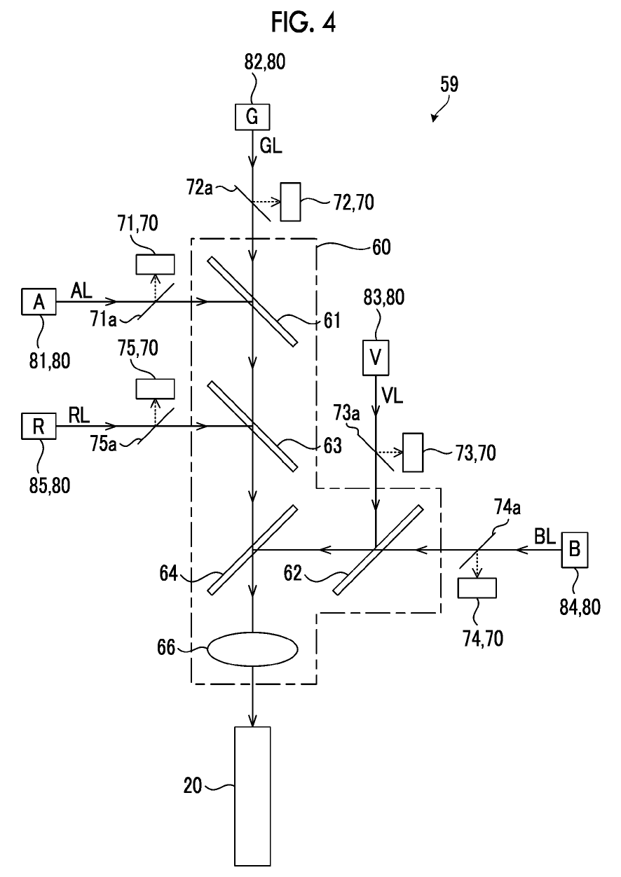

FIG. 4 is a schematic diagram showing an internal configuration example of the light source device 59 shown in FIG. 2. The light source device 59 is one aspect of a light source device, and comprises five light sources 80 that generate light of different colors, an optical member 60 that is configured to introduce the light generated by the five light sources 80 into the light guide 41 of the endoscope 10, five photodetector elements 70 that are configured to detect a portion of the light generated by each of the five light sources 80, and branch portions 71a, 72a, 73a, 74a, and 75a that branch the light. The five light sources 80 are controlled by the controller 58. The controller 58 controls the light amount of at least one of the five light sources 80 based on the light detected by the photodetector element 70.

The five light sources 80 generate light in five wavelength ranges having different peak wavelengths (wavelengths at which the intensity is maximum). The five light sources 80 include, for example, a B light source 84 that generates light in a blue wavelength range (hereinafter, referred to as B light), a V light source 83 that generates light in a violet wavelength range (hereinafter, referred to as V light), a G light source 82 that generates light in a green wavelength range (hereinafter, referred to as G light), an A light source 81 that generates light in an amber (or red) wavelength range (hereinafter, referred to as A light), and an R light source 85 that generates light in a red wavelength range (hereinafter, referred to as R light).

In FIG. 4, the B light is referred to as BL, the V light is referred to as VL, the G light is referred to as GL, the A light is referred to as AL, and the R light is referred to as RL. Each light source 80 includes a light-emitting element such as a light-emitting diode or a laser diode, and is configured to include a phosphor, an excitation light cut filter, and the like as necessary.

The photodetector element 70 is configured to include a light-receiving element such as a photodiode or a photoresistor. The five photodetector elements 70 include a B light detector element 74 that is capable of detecting a portion of the B light generated by the B light source 84, a V light detector element 73 that is capable of detecting a portion of the V light generated by the V light source 83, a G light detector element 72 that is capable of detecting a portion of the G light generated by the G light source 82, an A light detector element 71 that is capable of detecting a portion of the A light generated by the A light source 81, and an R light detector element 75 that is capable of detecting a portion of the R light generated by the R light source 85.

Since the five photodetector elements 70 are each configured with the same semiconductor material, the manufacturing costs of the light source device 59 can be reduced. As an example, the semiconductor material constituting the photoelectric conversion element included in the photodetector element 70 is silicon.

The branch portions 71a, 72a, 73a, 74a, and 75a are optical elements that branch (divide intensity of light) light, such as a half mirror and an optical coupler. In addition, a dichroic prism or the like may be used as the branch portions 71a, 72a, 73a, 74a, and 75a.

The optical member 60 includes, for example, dichroic mirrors 61 to 64 and a condenser lens 66 disposed between the dichroic mirror 64 and the light guide rod 20.

The A light source 81 emits the generated A light to the branch portion 71a. The branch portion 71a branches the A light emitted from the A light source 81 and emits each branched A light to the A light detector element 71 and the dichroic mirror 61. The A light detector element 71 receives the A light emitted from the branch portion 71a and outputs a signal indicating the intensity of the received A light.

The G light source 82 emits the generated G light to the branch portion 72a. The branch portion 72a branches the G light emitted from the G light source 82 and emits each branched G light to the G light detector element 72 and the dichroic mirror 61. The G light detector element 72 receives the G light emitted from the branch portion 72a and outputs a signal indicating the intensity of the received G light.

The V light source 83 emits the generated V light to the branch portion 73a. The branch portion 73a branches the V light emitted from the V light source 83 and emits each branched V light to the V light detector element 73 and the dichroic mirror 62. The V light detector element 73 receives the V light emitted from the branch portion 73a and outputs a signal indicating the intensity of the received V light.

The B light source 84 emits the generated B light to the branch portion 74a. The branch portion 74a branches the B light emitted from the B light source 84 and emits each branched B light to the B light detector element 74 and the dichroic mirror 62. The B light detector element 74 receives the B light emitted from the branch portion 74a and outputs a signal indicating the intensity of the received B light.

The R light source 85 emits the generated R light to the branch portion 75a. The branch portion 75a branches the R light emitted from the R light source 85 and emits each branched R light to the R light detector element 75 and the dichroic mirror 63. The R light detector element 75 receives the R light emitted from the branch portion 75a and outputs a signal indicating the intensity of the received R light.

The dichroic mirror 61 transmits the G light emitted from the branch portion 72a and emits the G light to the dichroic mirror 63. In addition, the dichroic mirror 61 reflects the A light emitted from the branch portion 71a and emits the A light to the dichroic mirror 63. That is, the light emitted from the dichroic mirror 61 to the dichroic mirror 63 includes the G light and the A light.

The dichroic mirror 62 reflects the V light emitted from the branch portion 73a and emits the V light to the dichroic mirror 64. In addition, the dichroic mirror 62 transmits the B light emitted from the branch portion 74a and emits the B light to the dichroic mirror 64. That is, the light emitted from the dichroic mirror 62 to the dichroic mirror 64 includes the V light and the B light.

The dichroic mirror 63 transmits the G light and the A light emitted from the dichroic mirror 61 and emits the G light and the A light to the dichroic mirror 64. In addition, the dichroic mirror 63 transmits the R light emitted from the branch portion 75a and emits the R light to the dichroic mirror 64. That is, the light emitted from the dichroic mirror 63 to the dichroic mirror 64 includes the G light, the A light, and the R light.

The dichroic mirror 64 transmits the G light, the A light, and the R light emitted from the dichroic mirror 63 and emits the G light, the A light, and the R light to the condenser lens 66. In addition, the dichroic mirror 64 reflects the V light and the B light emitted from the dichroic mirror 62 and emits the V light and the B light to the condenser lens 66. That is, the light emitted from the dichroic mirror 64 to the condenser lens 66 includes the G light, the A light, the V light, the B light, and the R light.

The condenser lens 66 condenses the light including the G light, the A light, the V light, the B light, and the R light emitted from the dichroic mirror 64 and causes the condensed light to be incident on the light guide rod 20.

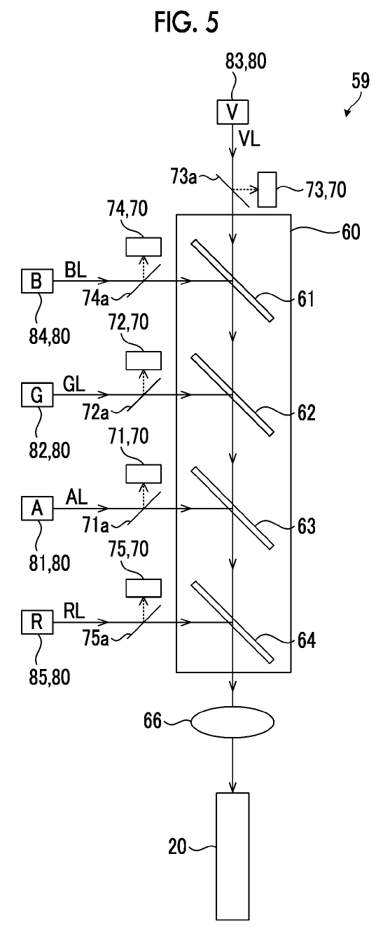

The position of each light source 80 is not limited to the position shown in FIG. 4 and can be arbitrarily determined. In addition, the configuration of the optical member 60 is not limited to the configuration shown in FIG. 4. The optical member 60 can also adopt other configurations (for example, see FIG. 5) as long as the optical member 60 is configured to introduce the A light, the B light, the V light, the G light, and the R light into the light guide 41.

The light source device 59 is operable in a plurality of modes in which the number of rays of light to be introduced into the light guide 41 of the endoscope 10 varies. The plurality of modes are selected according to the purpose in the endoscopy, and include, for example, a first mode in which A light, B light, G light, and V light are combined and introduced into the light guide 41, a second mode in which only R light is introduced into the light guide 41, and a third mode in which G light and V light are combined and introduced into the light guide 41.

The controller 58 controls the light amounts generated by at least two light sources of the five or more light sources 80 based on the light detected by the photodetector element 70. For example, in the first mode, the controller 58 performs control such that the amounts of the A light, the B light, the G light, and the V light are the target values. In addition, the controller 58 controls the ratio of a light amount of G light to V light in the third mode to be different from the ratio of a light amount of G light to V light in the first mode, based on the light detected by the G light detector element 72 and the V light detector element 73.

FIG. 5 is a schematic diagram showing another example of the internal configuration of the light source device 59 shown in FIG. 2. The light source device 59 may have a configuration shown in FIG. 5. In the configuration shown in FIG. 5, a portion different from the configuration shown in FIG. 4 will be described.

The branch portion 73a emits each of the branched V light to the V light detector element 73 and the dichroic mirror 61. The branch portion 74a emits each of the branched B light to the B light detector element 74 and the dichroic mirror 61. The branch portion 72a emits each of the branched G light to the G light detector element 72 and the dichroic mirror 62. The branch portion 71a emits each of the branched A light to the A light detector element 71 and the dichroic mirror 63. The branch portion 75a emits each of the branched R light to the R light detector element 75 and the dichroic mirror 64.

The dichroic mirror 61 transmits the V light emitted from the branch portion 73a and emits the V light to the dichroic mirror 62. In addition, the dichroic mirror 61 reflects the B light emitted from the branch portion 74a and emits the B light to the dichroic mirror 62. That is, the light emitted from the dichroic mirror 61 to the dichroic mirror 62 includes the V light and the B light.

The dichroic mirror 62 transmits the V light and the B light emitted from the dichroic mirror 61 and emits the V light and the B light to the dichroic mirror 63. In addition, the dichroic mirror 62 reflects the G light emitted from the branch portion 72a and emits the G light to the dichroic mirror 63. That is, the light emitted from the dichroic mirror 62 to the dichroic mirror 63 includes the V light, the B light, and the G light.

The dichroic mirror 63 transmits the V light, the B light, and the G light emitted from the dichroic mirror 62 and emits the V light, the B light, and the G light to the dichroic mirror 64. In addition, the dichroic mirror 63 reflects the A light emitted from the branch portion 71a and emits the A light to the dichroic mirror 64. That is, the light emitted from the dichroic mirror 63 to the dichroic mirror 64 includes the V light, the B light, the G light, and the A light.

The dichroic mirror 64 transmits the V light, the B light, the G light, and the A light emitted from the dichroic mirror 63 and emits the V light, the B light, the G light, and the A light to the condenser lens 66. In addition, the dichroic mirror 64 reflects the R light emitted from the branch portion 75a and emits the R light to the condenser lens 66. That is, the light emitted from the dichroic mirror 64 to the condenser lens 66 includes the V light, the B light, the G light, the A light, and the R light.

In the example of FIG. 4, the photodetector element 70 that detects a portion of the light generated by the light source 80 is provided in correspondence with each of the five light sources 80. However, the number of the photodetector elements 70 may be smaller than the number of the light sources 80. The photodetector element 70 may be provided only for the light source 80 for which the variable control of the light amount is required. For example, in a case where the A light is always controlled to a constant light amount, the A light detector element 71 may be omitted. As described above, depending on the control pattern of the light source 80, at least one of the B light detector element 74, the V light detector element 73, the G light detector element 72, or the A light detector element 71 can be omitted.

The light source device 59 comprises the five light sources 80, but may comprise six or more light sources 80 that generate light of different colors. In addition, the light source device 59 may comprise four or fewer light sources 80.

The G light and the B light are, for example, light having a peak wavelength of 400 [nm] or more and 580 [nm] or less, and have peak wavelengths different from each other. The G light and the B light are examples of first light. In this case, the G light source 82 and the B light source 84 are examples of first light sources, and the wavelength ranges of the G light and the B light are examples of first wavelength ranges.

The R light and the A light are, for example, light having a peak wavelength of 580 [nm] or more and 900 [nm] or less, and have peak wavelengths different from each other. Both the R light and the A light are light in a treatment light region that can be used as treatment light by PBM of a duodenum.

The R light is, for example, light having a peak wavelength of about 680 [nm], and is an example of second light. In this case, the R light source 85 is an example of a second light source, and the wavelength range of the R light is an example of a second wavelength range. The A light is, for example, light having a peak wavelength of about 590 [nm], and is an example of third light. In this case, the A light source 81 is an example of a third light source, and the wavelength range of the A light is an example of a third wavelength range.

As described above, the light source device 59 shown in FIG. 4 is a light source device comprising the G light source 82 and the B light source 84 (first light sources), the R light source 85 (second light source), and the A light source 81 (third light source).

### Hardware for Communication between Endoscope 10 and Control Device 11

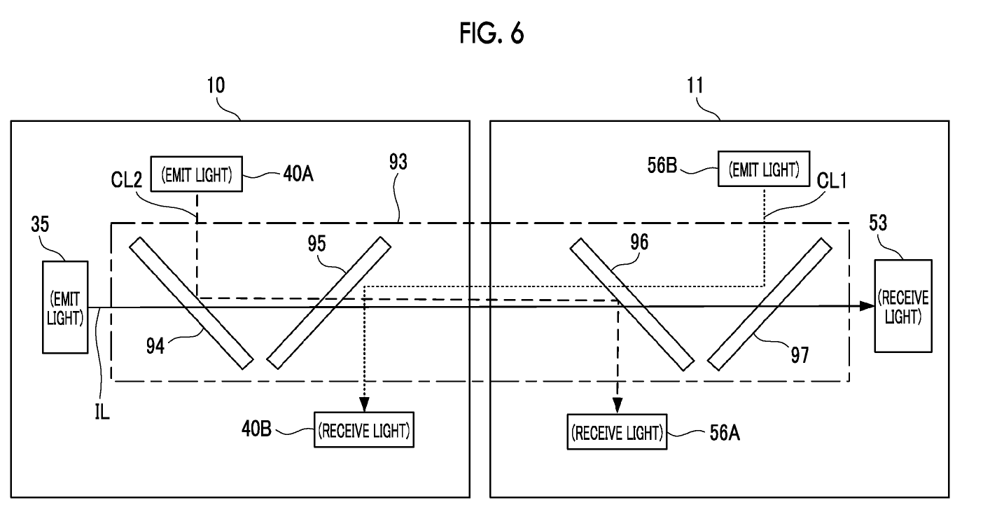

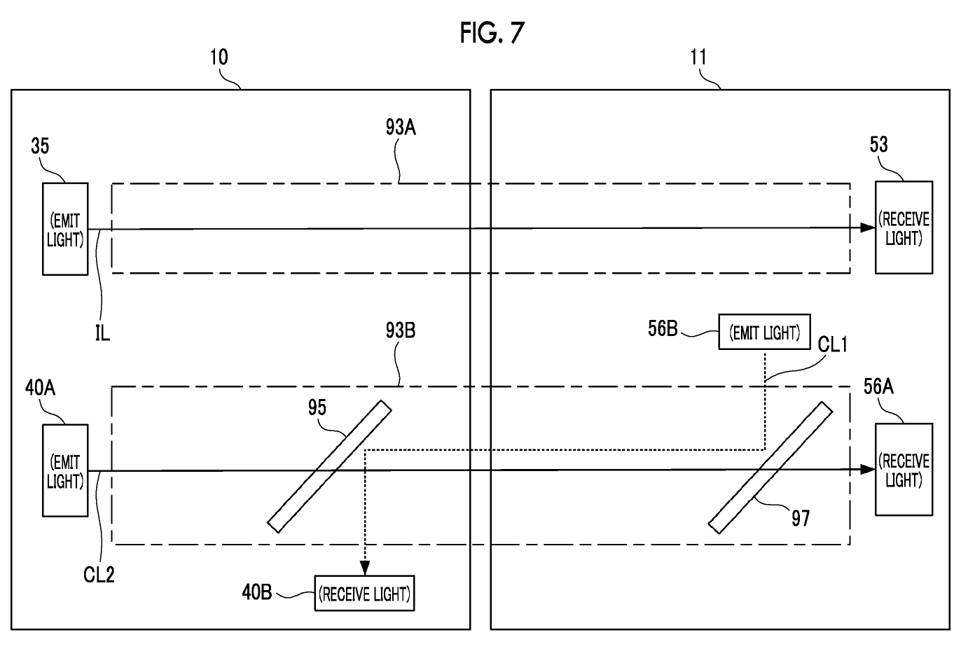

FIG. 6 is a schematic view showing an example of a hardware configuration for the endoscope 10 and the control device 11 to communicate with each other. The endoscope 10 and the control device 11 are provided with an optical path 93 through which light used for communication passes. The optical path 93 may be separately provided for an optical path used for communication of the image signal and an optical path used for communication of the control signal (for example, refer to FIG. 7). However, in the example of FIG. 6, two optical paths having different applications are unified into one optical path. That is, the optical path 93 serves as both an optical path of light based on the image signal and an optical path of light based on the control signals (the first control signal and the second control signal).

A dichroic mirror 94 and a dichroic mirror 95 are provided on the optical path 93 provided in the endoscope 10. A dichroic mirror 96 and a dichroic mirror 97 are provided on the optical path 93 provided in the control device 11.

The signal transmission and reception unit 40 of the endoscope 10 includes a light-emitting device 40A and a light-receiving device 40B. Light IL based on the image signal emitted from the image signal transmission unit 35 is incident on the dichroic mirror 94, is transmitted through the dichroic mirror 94, is incident on the dichroic mirror 95, and is transmitted through the dichroic mirror 95 to the control device 11.

Light CL2 based on the second control signal emitted from the light-emitting device 40A is incident on the dichroic mirror 94, is reflected by the dichroic mirror 94, is incident on the dichroic mirror 95, and is transmitted through the dichroic mirror 95 to the control device 11.

The signal transmission and reception unit 56 of the control device 11 includes a light-emitting device 56B and a light-receiving device 56A. The light IL based on the image signal transmitted through the dichroic mirror 95 of the endoscope 10 is incident on the dichroic mirror 96, is transmitted through the dichroic mirror 96, is incident on the dichroic mirror 97, is transmitted through the dichroic mirror 97, and is incident on the image signal reception unit 53.

The light CL2 based on the second control signal transmitted through the dichroic mirror 95 of the endoscope 10 is incident on the dichroic mirror 96, is reflected from the dichroic mirror 96, and is incident on the light-receiving device 56A.

Light CL1 based on the first control signal emitted from the light-emitting device 56B is incident on the dichroic mirror 97, is reflected from the dichroic mirror 97, is incident on the dichroic mirror 96, is transmitted through the dichroic mirror 96, and is incident on the dichroic mirror 95 of the endoscope 10. The light CL1 based on the first control signal incident on the dichroic mirror 95 is reflected from the dichroic mirror 95 and is incident on the light-receiving device 40B.

The optical path 93, the dichroic mirror 94, the dichroic mirror 95, the light-emitting device 40A, the light-receiving device 40B, the image signal transmission unit 35, the image signal modulation unit 34, the signal conversion unit 39, the dichroic mirror 96, the dichroic mirror 97, the light-receiving device 56A, the light-emitting device 56B, the image signal reception unit 53, the image signal demodulation unit 54, and the signal conversion unit 57 constitute a communication unit that performs optical communication of the image signal and the control signal between the endoscope 10 and the control device 11.

The image signal has a larger data amount than the control signal, and a high transfer rate is also required. Therefore, the communication frequency (modulation frequency) of the image signal is set to be higher than the communication frequency (modulation frequency) of the first control signal and the second control signal. In consideration of such a difference in communication frequency, it is preferable that the light-emitting device of the image signal transmission unit 35 used for communication of the image signal and the light-emitting device 40A and the light-emitting device 56B used for communication of the control signal are different types.

For example, by using a laser diode as the light-emitting device included in the image signal transmission unit 35, high-speed data transfer is facilitated. In addition, by using a light-emitting diode as the light-emitting device 40A and the light-emitting device 56B, it is possible to reduce the manufacturing costs. Since the image signals can be transferred at a high speed, responsiveness of the AE function based on the image signals transferred at a high speed can be improved. As a result, the light amount control can be performed with higher accuracy by the light amount control of the light source 80 and the AE function based on the output of the photodetector element 70.

FIG. 7 is a schematic diagram showing another example of the hardware configuration for the endoscope 10 and the control device 11 to perform communication. In FIG. 7, a configuration in which an optical path 93A used for the communication of the image signal and an optical path 93B used for the communication of the control signal are separately provided will be described.

In the optical path 93A, the light IL based on the image signal emitted from the image signal transmission unit 35 is incident on the image signal reception unit 53 without passing through the dichroic mirror.

The dichroic mirror 95 is provided in the optical path 93B provided in the endoscope 10. The dichroic mirror 97 is provided in the optical path 93B provided in the control device 11.

The light CL2 based on the second control signal emitted from the light-emitting device 40A is incident on the control device 11 by being transmitted through the dichroic mirror 95. The light CL2 incident on the control device 11 is incident on the light-receiving device 56A by being transmitted through the dichroic mirror 97.

The light CL1 based on the first control signal emitted from the light-emitting device 56B is incident on the endoscope 10 by being reflected by the dichroic mirror 97. The light CL1 incident on the endoscope 10 is incident on the light-receiving device 40B by being reflected by the dichroic mirror 95. Further, a configuration may be adopted in which the light-emitting device 40A and the light-receiving device 56A, and the light-emitting device 56B and the light-receiving device 40B are disposed to face each other, and the dichroic mirror is not used.

In the configuration shown in FIG. 7, it is preferable that the light-emitting device included in the image signal transmission unit 35 is a laser diode, and that the light-emitting device 40A and the light-emitting device 56B are light-emitting diodes. As a result, it is possible to reduce the manufacturing costs while facilitating high-speed data transfer for the image signal.

### Observation Mode and Treatment Mode

The control device 11 can be operated in an observation mode and a treatment mode. The observation mode is used, for example, for observing the duodenum in the treatment of diabetes. The treatment mode is used, for example, for irradiating the duodenum with treatment illumination light in the treatment of diabetes by PBM. The observation mode and the treatment mode are switched by, for example, an operation from a user (for example, a doctor).

In the observation mode, the control device 11 emits illumination light including the G light and the B light (first light) output from at least the G light source 82 and the B light source 84 (first light source) from the endoscope 10 to the subject, and outputs a first image generated from the image signal obtained in that state. For example, the control device 11 displays the first image on the monitor 19. As a result, the user can observe the state of the subject. The illumination light emitted in the observation mode is referred to as observation illumination light. The observation illumination light is an example of first illumination light.

In addition, in the observation mode, the control device 11 may further turn on the A light source 81. That is, the observation illumination light may include the A light (third light) in addition to the G light and the B light (first light). Although a configuration in which the light source device 59 includes the third light source that outputs the third light in the third wavelength range has been described, the third light source may be included in an irradiation instrument different from the light source device 59.

In addition, in the observation mode, the control device 11 may turn on the R light source 85 (second light source) in addition to the G light source 82 and the B light source 84 (first light source). That is, the observation illumination light may include the R light (second light) in addition to the G light and the B light (first light). In this way, by using the R light source 85 (second light source) used for the treatment illumination light in the treatment mode for the observation illumination light in the observation mode, it is not necessary to provide a red light source for the observation illumination light separately from the R light source 85, and the configuration can be simplified.

In the treatment mode, the control device 11 emits the illumination light including the R light (second light) output from the R light source 85 (second light source) to the subject. For example, by irradiating the duodenum with the illumination light, it is possible to perform the treatment for diabetes by PBM. The illumination light emitted in the treatment mode is referred to as treatment illumination light. The treatment illumination light is an example of second illumination light.

In addition, in the treatment mode, the control device 11 may output the second image generated from the image signal obtained in a state in which the treatment illumination light is emitted to the subject. For example, the control device 11 displays the second image on the monitor 19. As a result, the user can observe the state of the subject while performing the treatment by irradiation with the treatment illumination light.

In addition, in the treatment mode, the control device 11 may turn on the G light source 82 and the B light source 84 (first light source) in addition to the R light source 85 (second light source). That is, the treatment illumination light may include the G light and the B light (first light) in addition to the R light (second light). As a result, in a case where the user observes the state of the subject in the treatment mode as described below, a color image that is easy to visually recognize can be obtained as a display image. In addition, in the treatment mode, the control device 11 may further turn on the V light source 83. In this case, the treatment illumination light includes, for example, the G light, the B light, the R light, and the V light.

In addition, in the treatment mode, the control device 11 may further turn on the A light source 81. In this case, the treatment illumination light includes, for example, the G light, the B light, the R light, the V light, and the A light. As described above, the A light is the light in the treatment light region together with the R light, and the treatment effect can be enhanced by irradiating the A light in addition to the R light.

As described in FIG. 4, the light source device 59 comprises the R light source 85 (second light source) used in the treatment mode in addition to the G light source 82 and the B light source 84 (first light source) used in the observation mode, and the control device 11 controls the light amounts of these light sources. The control device 11 performs different controls on the light amounts of the G light source 82 and the B light source 84 (first light source) between the observation mode and the treatment mode.

### Wavelength Spectrum of Observation Illumination Light in Observation Mode

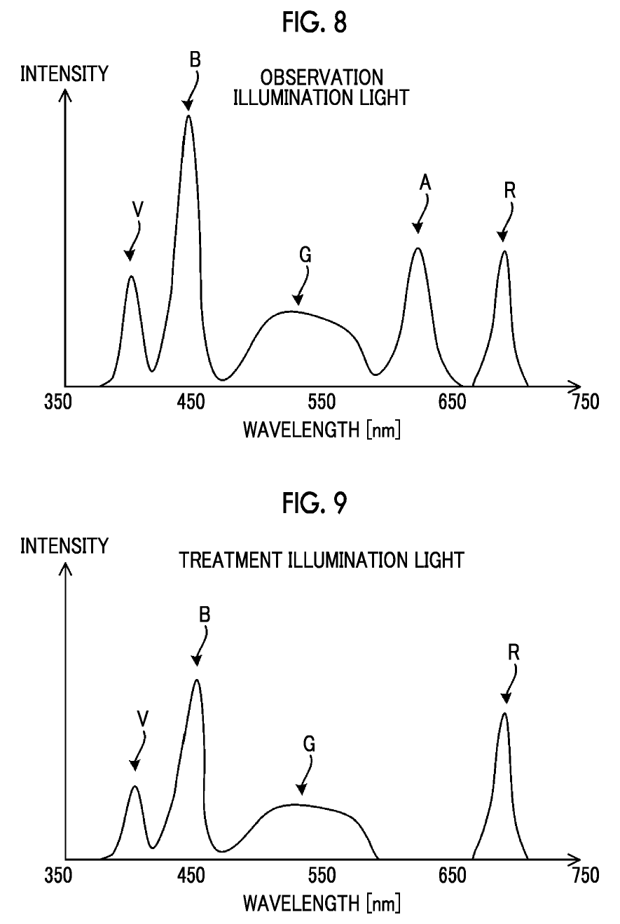

FIG. 8 is a diagram showing an example of a wavelength spectrum of the observation illumination light in the observation mode. FIG. 9 is a diagram showing an example of a wavelength spectrum of the treatment illumination light in the treatment mode. In FIGS. 8 and 9, the B light is indicated by B, the G light is indicated by G, the V light is indicated by V, the A light is indicated by A, and the R light is indicated by R.

As shown in FIG. 8, the observation illumination light in the observation mode includes, for example, the V light, the B light, the G light, the A light, and the R light. The observation illumination light is, for example, white light. As shown in FIG. 9, the treatment illumination light in the treatment mode includes, for example, the V light, the B light, the G light, and the R light.

It is preferable that a ratio of a light amount of the R light (second light) to light amounts of the G light and the B light (first light) in the treatment illumination light is larger than a ratio of a light amount of the R light (second light) to light amounts of the G light and the B light (first light) in the observation illumination light.

It is assumed that a light amount of the G light is g, a light amount of the B light is b, and a light amount of the R light is r. For example, (r/g in the treatment mode) > (r/g in the observation mode) is established. In addition, (r/b in the treatment mode) > (r/b in the observation mode) may be established. In the example of FIG. 8, (r/g in the treatment mode) > (r/g in the observation mode) and (r/b in the treatment mode) > (r/b in the observation mode) are established.

As described above, the control device 11 turns on the G light source 82 and the B light source 84 (first light source) not only in the observation mode but also in the treatment mode, and performs different controls on the light amounts of the G light source 82 and the B light source 84 (first light source) between the observation mode and the treatment mode.

For example, the control device 11 can cause the user to observe the state of the subject by turning on the G light source 82 and the B light source 84 (first light source) even in the treatment mode to perform the treatment with the PBM and displaying the second image generated from the image signal obtained in that state on the monitor 19.

Here, it is desirable that the light amount of each color (for example, the G light, the B light, and the R light) in the observation illumination light in the observation mode is set to a ratio suitable for the observation. However, in a case where the light amount of each color is set in the same manner in the treatment illumination light in the treatment mode, the light amount of the R light (second light) may be insufficient, and the effect of the treatment may not be sufficiently obtained.

Therefore, as described above, the ratio of the light amount of the R light (second light) to the light amounts of the G light and the B light (first light) in the treatment illumination light is set to be larger than the ratio of the light amount of the R light (second light) to the light amounts of the G light and the B light (first light) in the observation illumination light. As a result, in the observation mode, the observation can be accurately performed with the image suitable for the observation, and in the treatment mode, the observation of the state of the subject can be performed while the treatment with the PBM is performed.

As described above, the G light source 82 and the B light source 84 (first light source) are turned on not only in the observation mode but also in the treatment mode, and different controls are performed on the light amounts of the G light source 82 and the B light source 84 (first light source) between the observation mode and the treatment mode, so that the observation and the treatment can be appropriately performed.

In addition, as in the examples of FIGS. 8 and 9, in the treatment mode, the control device 11 may cause the R light (second light) to emit light and cause the A light (third light) not to emit light, among the R light (second light) and the A light (third light) which are the red light. As a result, in the treatment for which the wavelength range of the R light is suitable, the R light is caused to emit light and the A light is caused not to emit light, so that more appropriate treatment can be performed. However, in the treatment mode, the control device 11 may cause only the A light to emit light among the R light and the A light, or may cause both the R light and the A light to emit light.

### Irradiation with Observation Illumination Light and Treatment Illumination Light

The observation illumination light and the treatment illumination light are emitted from the illumination window 42 shown in FIG. 4. In a case where a plurality of the illumination windows 42 are provided, the observation illumination light and the treatment illumination light may be configured to be emitted from the plurality of the same illumination windows 42, or the observation illumination light and the treatment illumination light may be configured to be emitted from different illumination windows 42.

### Red Pixel Suppression

In the treatment mode, since the ratio of the light amount of the R light (second light) to the light amount of the G light and the B light (first light) is large as described above, the second image generated from the image signal obtained by the imaging may be reddish. Therefore, in the treatment mode, the control device 11 may perform image processing of suppressing the brightness of the red component on the image signal obtained by the imaging, compared to the case of the observation mode. As a result, the second image displayed in the treatment mode can be an image in which the brightness of the red color is suppressed compared to the first image displayed in the observation mode. As a result, the color of the second image displayed in the treatment mode can be brought closer to the color of the first image displayed in the observation mode, and the visibility in the treatment mode can be improved.

In a configuration in which the G light and the B light (first light) are not emitted in the treatment mode, a configuration may be adopted in which the image processing of suppressing the brightness of the red component is not performed on the image signal obtained by the imaging in the treatment mode.

### Gain Suppression

In the treatment mode, the control device 11 may perform image processing of suppressing the gain for the image signal obtained by the imaging, compared to the case of the observation mode. As a result, the second image displayed in the treatment mode can be an image generated with a smaller gain than the first image displayed in the observation mode. As a result, in the treatment mode, it is possible to suppress a decrease in the visibility of the second image due to a large amount of light.

### Emission Range of Observation Illumination Light and Treatment Illumination Light

FIG. 10 is a diagram showing an example of an emission range of the observation illumination light and the treatment illumination light. FIG. 10 shows a state in which the insertion part 13 is inserted into a duodenum 200. In this state, the observation illumination light (observation mode) or the treatment illumination light (treatment mode) is emitted from the illumination window 42 of the tip part 14 of the insertion part 13, and the imaging is performed by the imaging unit 30 provided in the tip part 14.

An emission range 211 shows an emission range of the observation illumination light and the treatment illumination light from the illumination window 42. That is, the observation illumination light or the treatment illumination light is emitted to a portion included in the emission range 211 of an inner surface of the duodenum 200.

An imaging range 212 shows an imaging range (angle of view) of the imaging unit 30. That is, a portion included in the imaging range 212 of the inner surface of the duodenum 200 is imaged and displayed as an endoscopic image 110.

The observation illumination light and the treatment illumination light are diffused light instead of the laser light (collimated light). As a result, as shown in FIG. 10, the emission range 211 is wider than the imaging range 212. In this way, it is preferable that the observation illumination light and the treatment illumination light are emitted in a range wider than the imaging range 212 of the endoscope 10. The range wider than the imaging range 212 is, for example, a range including the imaging range 212 and wider than the imaging range 212.

For example, in photodynamic therapy (PDT), the laser light is used as the treatment light, but in the treatment using PBM, the diffused light is used as the treatment illumination light, so that the lumen of the subject can be uniformly irradiated with the treatment illumination light, and the treatment using PBM can be appropriately performed.

### Endoscopic Image Displayed on Monitor 19

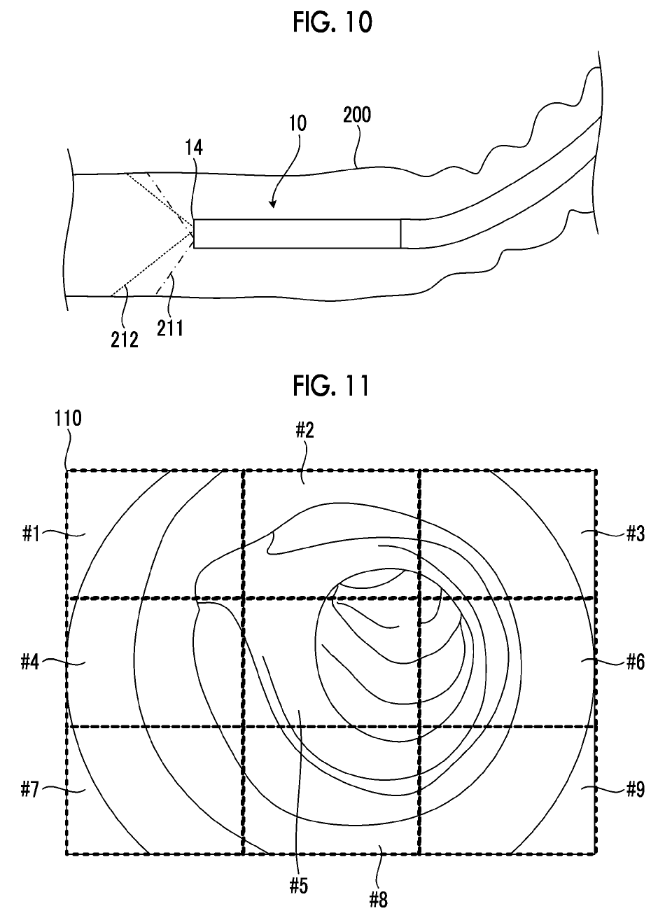

FIG. 11 is a diagram showing an example of the endoscopic image displayed on the monitor 19. In the treatment mode, for example, the endoscopic image 110 is displayed on the monitor 19. The endoscopic image 110 is a second image based on the image signal obtained by the imaging of the insertion part 13 inserted into the duodenum by the imaging unit 30 in the treatment mode.

In the endoscopic image 110, divided regions #1 to #9 obtained by dividing the endoscopic image 110 into nine regions are set. The boundary lines of the divided regions #1 to #9 are indicated by thick dotted lines in the drawing, but may be displayed as an image or may not be displayed as an image. In addition, in FIG. 11, the example in which the endoscopic image 110 is divided into nine divided regions #1 to #9 has been described, but the number of divisions of the endoscopic image 110 is not limited to nine and can be set to any number.

### Guidance Display in Endoscopic Image 110

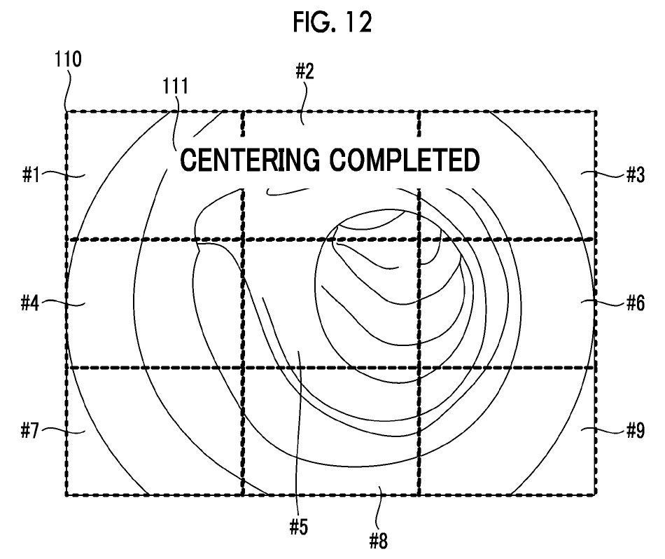

FIG. 12 is a diagram showing an example of the guidance display in the endoscopic image 110. In a state in which the insertion part 13 is inserted into the duodenum, in a case in which the tip part 14 is deviated from the center of the lumen of the duodenum, luminance bias occurs in the endoscopic image 110. For example, in a case in which the tip part 14 is deviated to the upper right in a case of being viewed in an axial direction of the lumen of the duodenum, the upper right region (for example, the regions of the divided regions #2, #3, and #6) of the endoscopic image 110 is darkened. The user operates the operation unit 15 based on this to perform centering of bringing the position of the tip part 14 close to the center of the lumen of the duodenum.

In a case in which the position of the tip part 14 is brought close to the center of the lumen of the duodenum, the control device 11 may display a guidance display 111 in a superimposed manner on the endoscopic image 110. The guidance display 111 is a character string "CENTERING COMPLETED" indicating that the centering is completed. As a result, the user can easily recognize that the centering is completed.

For example, the control device 11 determines whether or not the position of the tip part 14 is close to the center of the lumen of the duodenum based on the image signal obtained by the imaging unit 30 of the endoscope 10. For example, the control device 11 determines whether or not the position of the tip part 14 is close to the center of the lumen of the duodenum based on the bias of the average value of the luminance for each region (for example, for each divided region) of the image signal. A method of determining whether or not the position of the tip part 14 is close to the center of the lumen of the duodenum is not limited to the method based on the image signal, and may be a method based on information obtained by another sensor provided in the tip part 14.

The guidance display 111 is an example of guide information for guiding the position of the insertion part 13 for emitting the treatment illumination light. The output of the guide information is not limited to the display of the guidance display 111, and may be a display of an icon or the like, a change in the color of the frame image of the endoscopic image 110, a voice output, or the like.

In addition, the guide information is not limited to the information indicating that the centering is completed, and may be information for guiding the operation direction or the operation amount for performing the centering of the insertion part 13 before the centering is completed.

### Calculation of Irradiation Energy

In the treatment mode, the control device 11 may calculate and output the irradiation energy of the R light (second light) in the treatment region from which the treatment illumination light is emitted. For example, the control device 11 calculates the irradiation energy of the R light (second light) in the treatment region based on the distance information indicating the distance from the tip part of the insertion part 13 to the treatment region and the light amount information indicating the light amount of the R light (second light). In addition, for example, the control device 11 displays information indicating the calculated irradiation energy on the monitor 19. As a result, the user can perform the treatment using the PBM while checking the irradiation energy of the treatment illumination light, which is important in the treatment using the PBM.

The irradiation energy is illuminance (W/cm²), and can be calculated based on the light amount information and the distance information by using a relationship in which the illuminance is inversely proportional to the square of the distance.

Regarding the light amount information, the control device 11 acquires the light amount information based on, for example, the drive current value of the R light source 85 (second light source). Alternatively, the control device 11 may calculate the light amount information based on the detection value obtained by the photodetector element 70 (for example, the R light detector element 75) of the second light source. Alternatively, the control device 11 may calculate the light amount information based on the image signal obtained by the image signal demodulation unit 54. Alternatively, the control device 11 may calculate the light amount information based on a plurality of pieces of information including the drive current value of the R light source 85 (second light source), the detection value obtained by the photodetector element 70 (for example, the R light detector element 75) of the second light source, and the image signal obtained by the image signal demodulation unit 54.

Regarding the distance information, for example, the distance information is stored in the memory of the control device 11, and the control device 11 acquires the distance information from the memory. As an example, for each combination of the insertion target (for example, duodenum) of the insertion part 13 and the model of the insertion part 13, correspondence information indicating the distance information in a case where the insertion part 13 is inserted into the insertion target is stored in the memory of the control device 11, and the control device 11 acquires the distance information by using the correspondence information. Alternatively, a distance-measuring sensor may be provided at the tip part of the insertion part 13, and the control device 11 may acquire the distance information based on the detection value obtained by the distance-measuring sensor. Alternatively, the control device 11 may acquire the distance information by image recognition based on the image signal obtained by the image signal demodulation unit 54. Alternatively, the control device 11 may acquire the distance information by receiving an input of the distance from the user.

In addition, the control device 11 may calculate the integrated irradiation energy of the R light (second light) based on the calculated irradiation energy of the R light (second light) and the emission time of the R light (second light), and may output the calculated integrated irradiation energy. For example, the control device 11 displays information indicating the calculated integrated irradiation energy on the monitor 19. As a result, the user can perform the treatment using the PBM while checking the integrated irradiation energy of the treatment illumination light, which is important in the treatment using the PBM.

For example, in a case where the integrated irradiation energy is denoted by E, the irradiation energy is denoted by P, and the emission time is denoted by T, the control device 11 calculates the integrated irradiation energy by E = P x T [J/cm²].

### Notification of Integrated Irradiation Energy for Each Division Region

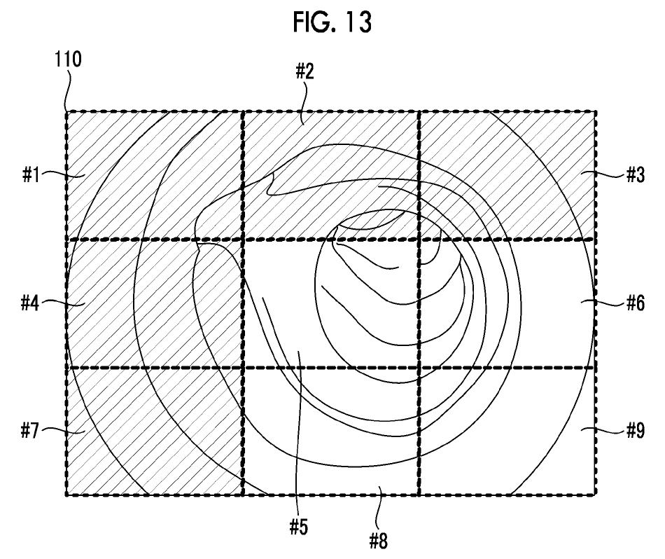

FIG. 13 is a diagram showing an example of a notification of the integrated irradiation energy for each division region. The control device 11 may calculate the irradiation energy or the integrated irradiation energy for each of the division regions #1 to #9. For example, the control device 11 acquires the distance information for each of the division regions #1 to #9, and calculates the irradiation energy or the integrated irradiation energy for each of the division regions #1 to #9.

As an example, the control device 11 may calculate the integrated irradiation energy for each of the division regions #1 to #9, and notify the user of the division region in which the calculated integrated irradiation energy has reached a predetermined value. For example, as shown in FIG. 13, the control device 11 changes the color of the division region (in the example of FIG. 13, the division regions #1 to #3, #4, and #7) in which the integrated irradiation energy has reached the predetermined value among the division regions #1 to #9, to notify the user of the division region in which the integrated irradiation energy has reached the predetermined value. As a result, the user can easily recognize the region in which the integrated irradiation energy has reached the predetermined value among the regions in the duodenum shown in the endoscopic image 110.

The predetermined value to be compared with the integrated irradiation energy may be set in advance or may be selected by the user.

### Stopping of AE Function in Treatment Mode

In the observation mode, the control device 11 enables the AE function of controlling the luminance of the observation illumination light (for example, the G light and the B light) based on the luminance information calculated from the image signal as described above. On the other hand, in the treatment mode, the control device 11 does not control the luminance of the treatment illumination light based on the luminance information calculated from the image signal. In the treatment using the PBM, the optimization of the irradiation energy or the integrated irradiation energy is prioritized over the optimization of the luminance of the display image. Therefore, in the treatment mode, the appropriate treatment can be performed by controlling the luminance of the R light (second light) regardless of the luminance information calculated from the image signal.

### Switching of Observation Illumination Light and Treatment Illumination Light at Predetermined Interval

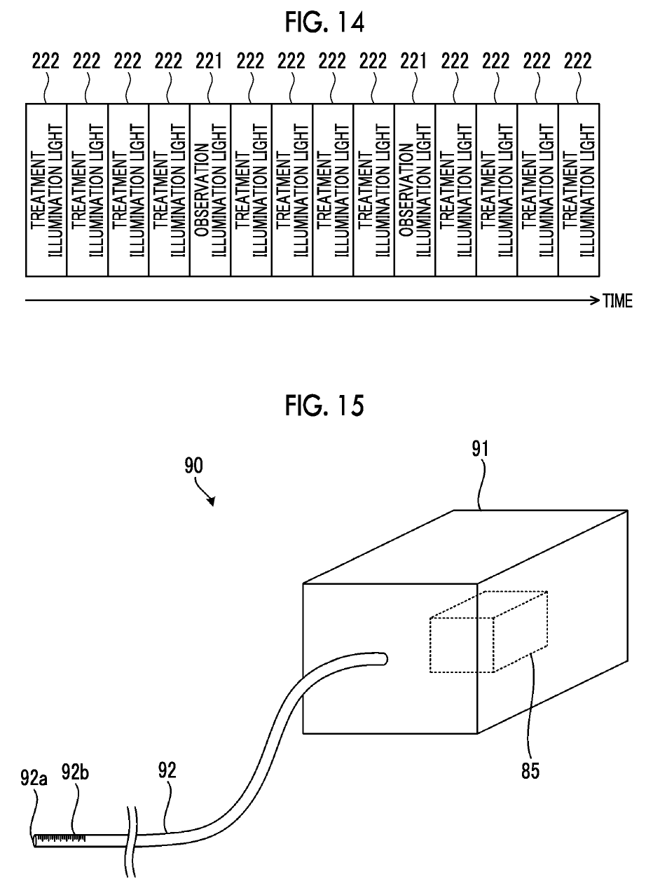

FIG. 14 is a diagram showing an example of switching between the observation illumination light and the treatment illumination light at a predetermined interval. In the treatment mode, the control device 11 may repeatedly switch between observation illumination light 221 and treatment illumination light 222 at a predetermined interval and display a third image generated from the image signal captured during the emission of the observation illumination light 221 on the monitor 19.

In the example of FIG. 14, an operation of emitting the treatment illumination light 222 for four frames and then emitting the observation illumination light 221 for one frame is repeated. Even with such a configuration, the user can observe the state of the subject while performing the treatment by emitting the treatment illumination light 222.

In the example of FIG. 14, the control of performing the operation of emitting the observation illumination light 221 for one frame immediately after emitting the treatment illumination light 222 for four frames without an interval has been described, but the present disclosure is not limited to such a configuration. For example, the period of emitting the treatment illumination light 222 is not limited to four frames and may be one to three frames or five or more frames. In addition, a period of performing another control or a standby period may be provided between the period of emitting the observation illumination light 221 and the period of emitting the treatment illumination light 222.

### Separate Configuration of Irradiation Instrument for Treatment Illumination Light

A configuration in which the light source device 59 comprises the R light source 85 (second light source) has been described, but the present disclosure is not limited to such a configuration. For example, the endoscope system 2 may comprise an irradiation instrument for treatment illumination light separately from the light source device 59.

FIG. 15 is a diagram showing an example of an irradiation instrument for treatment illumination light different from the light source device 59. An irradiation instrument 90 shown in FIG. 15 includes the R light source 85 (second light source) and a long probe 92 that emits the treatment illumination light including the R light source 85 (second light source) from the tip part of the insertion part 13. The R light source 85 is built in a housing 91 of the irradiation instrument 90. In this configuration, the light source device 59 need not comprise the configuration (see FIG. 4) of the R light source 85, the R light detector element 75 related to the R light source 85, the dichroic mirror 62, and the like.

The probe 92 is connected to the housing 91 and can emit the treatment illumination light including the R light source 85 (second light source) output by the R light source 85 from a tip part 92a of the probe 92. By inserting the probe 92 into the treatment tool channel 16a from the treatment tool insertion port 16 of the endoscope 10 shown in FIG. 1, the tip part 92a of the probe 92 can be exposed from the opening portion of the distal end surface of the insertion part 13.

For example, by inserting the insertion part 13 into the duodenum, inserting the probe 92 into the treatment tool channel 16a, and outputting the treatment illumination light from the irradiation instrument 90 in a state where the tip part 92a of the probe 92 is exposed from the opening portion of the distal end surface of the insertion part 13, the inside of the duodenum can be irradiated with the treatment illumination light.

The irradiation with the treatment illumination light by the irradiation instrument 90 may be controlled by the control device 11 communicating with the irradiation instrument 90, or may be controlled by a user operation via an operation unit provided in the irradiation instrument 90.

For example, in the observation mode, the observation illumination light output by the light source device 59 is emitted from the illumination window 42 provided in the tip part 14 of the insertion part of the endoscope 10. In addition, in the treatment mode, the treatment illumination light output by the irradiation instrument 90 is emitted from the tip part 92a of the probe 92 inserted from the treatment tool channel 16a of the endoscope 10. The observation illumination light emitted in the observation mode may further include light output by the irradiation instrument 90. The treatment illumination light emitted in the treatment mode may further include light output by the light source device 59.

In the configuration in which the irradiation instrument 90 for the treatment illumination light is provided separately from the light source device 59 as shown in FIG. 15, the control device 11 acquires the light amount information of the R light (second light) from, for example, the irradiation instrument 90 in the calculation of the irradiation energy of the R light (second light). In addition, in this case, the distance information may be information indicating a distance between the tip part of the irradiation instrument 90 and the treatment region, or may be information indicating a distance between the tip part of the insertion part 13 and the treatment region.

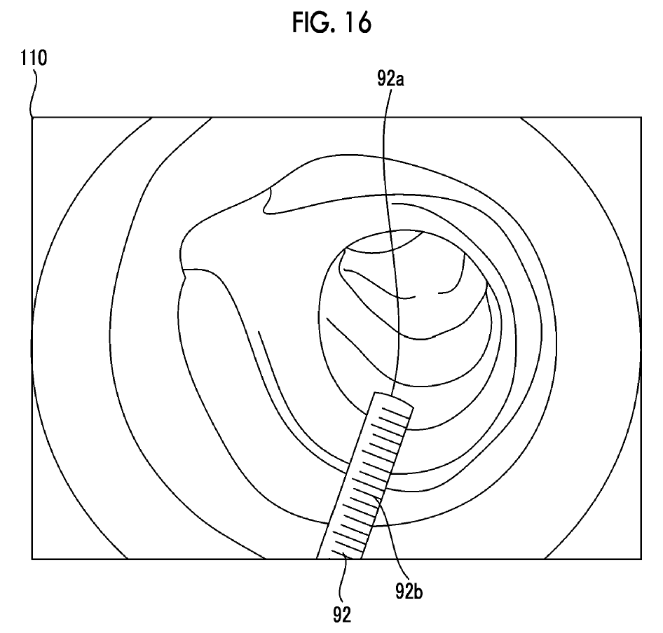

In addition, a scale 92b engraved at regular intervals may be provided in the vicinity of the tip part 92a of the probe 92. As a result, in a case where the tip part 92a protrudes from the opening portion of the distal end surface of the insertion part 13, the scale 92b is reflected in the endoscopic image 110 (second image), and the protrusion amount of the tip part 92a can be easily ascertained, and, for example, the irradiation amount control in a depth direction can be performed more precisely. FIG. 16 is a diagram showing an example of the scale 92b of the probe 92 reflected in the endoscopic image 110.

### Retention Mechanism for Retaining Position of Tip Part 14 of Insertion Part 13 of Endoscope 10 in Lumen of Subject

FIG. 17 is a diagram showing an example of a retention mechanism for retaining the position of the tip part 14 of the insertion part 13 of the endoscope 10 in the lumen of the subject. A balloon 300 may be provided in the vicinity of the tip part 14 of the endoscope 10. The balloon 300 is a retention mechanism for retaining the position of the tip part 14 of the insertion part 13 of the endoscope 10 in the lumen (for example, duodenum) of the subject.

For example, the balloon 300 is configured to be inflated by supplying air to the balloon 300 from the balloon connector 25 shown in FIG. 3 via the tube in response to the operation of the user on the operation unit 15. The balloon 300 is formed to have a symmetrical shape with respect to an axis of the insertion part 13 in the inflated state. Therefore, in a case where the balloon 300 is inflated, the tip part 14 of the insertion part 13 is retained at a substantially central position in the lumen of the subject.

As a result, the user can easily center the tip part 14 of the insertion part 13 in the lumen of the subject and retain the state. In addition, folds in the lumen of the subject can be stretched by the inflation of the balloon connector 25, and the treatment illumination light can be more evenly emitted.

### Other Examples of Observation Illumination Light and Treatment Illumination Light

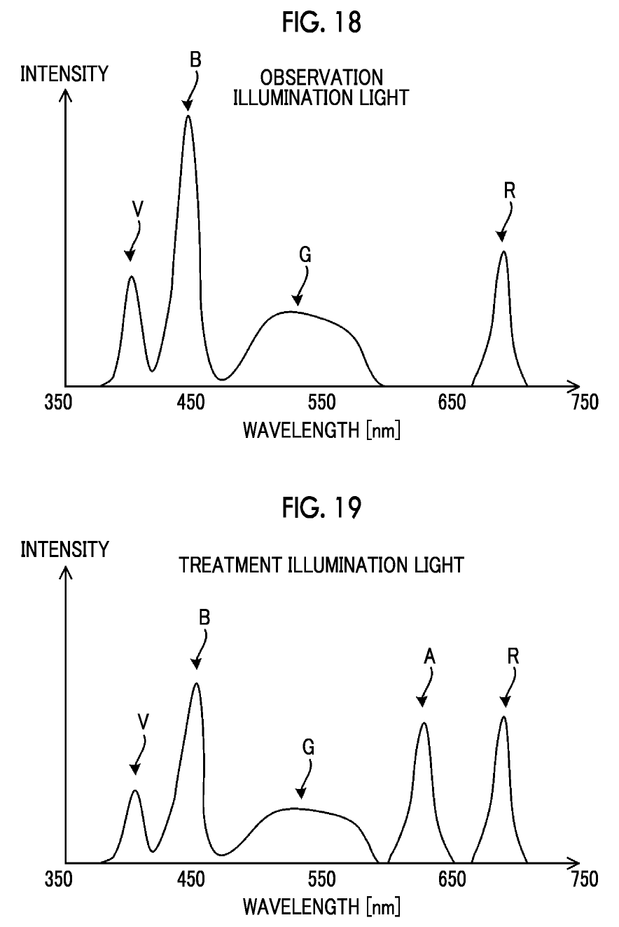

FIG. 18 is a diagram showing another example of the wavelength spectrum of the observation illumination light in the observation mode. FIG. 19 is a diagram showing another example of the wavelength spectrum of the treatment illumination light in the treatment mode. As shown in FIGS. 18 and 19, the observation illumination light may be light including V light, B light, G light, and R light and not including A light, and the treatment illumination light may be light including V light, B light, G light, A light, and R light.

That is, the control device 11 may be configured to cause only the R light source 85 (second light source) to emit light out of the R light source 85 (second light source) and the A light source 81 (third light source) in the observation mode, and to cause both the R light source 85 (second light source) and the A light source 81 (third light source) to emit light in the treatment mode.

As described above, in the treatment mode, by adding the red light source as compared with the observation mode, for example, even in a case where the maximum light amount that can be output by the R light source 85 is not large, the light amount of the treatment illumination light required for the PBM treatment can be sufficiently secured by using the A light source 81 in combination.

### Other Examples of Division Regions of Endoscopic Image 110

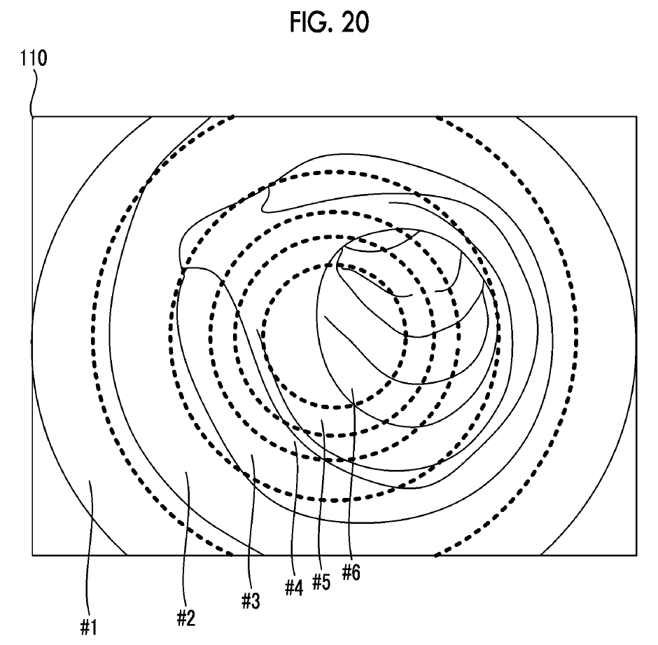

FIG. 20 is a diagram showing another example of the division regions of the endoscopic image 110. Although the division regions #1 to #9 obtained by dividing the endoscopic image 110 in a matrix shape have been described in FIG. 11, the method of dividing the endoscopic image 110 is not limited to the method of dividing the endoscopic image 110 in a matrix shape, and may be, for example, a method of dividing the endoscopic image 110 in a concentric circular shape. In the example shown in FIG. 20, the concentric circular division regions #1 to #6 are set in the endoscopic image 110. The division regions #1 to #5 excluding the innermost division region #6 have a hollow circular shape (donut shape). Since the duodenum has a lumen, it is possible to perform more accurate treatment by setting the concentric circular division regions.

### Modification Example 1

Although the G light and the B light have been described as an example of the first light, the first light may be any one of the G light or the B light.

### Modification Example 2

Although the R light has been described as an example of the second light and the A light has been described as an example of the third light, the second light may be the A light and the third light may be the R light.

### Modification Example 3

Although the configuration in which the light source device 59 is provided in the control device 11 has been described, the light source device 59 may be a device different from the control device 11.

In the present embodiment, each type of processing is executed by any computer. In addition, any computer may execute these processes using a processor as hardware, a program as software, or a combination thereof. In such a case, the processor is configured to execute various types of processing in the present embodiment in cooperation with the program, and may function as each unit or each means in the present embodiment. Further, the execution order of the processes by the processor is not limited to the order described and may be changed as appropriate. Any computer may be a general-purpose computer, a computer for a specific use, a workstation, or another system capable of executing each process.

The processor may be configured by one or more hardware components, and the types of hardware are not limited. For example, the processor may be configured with hardware such as a central processing unit (CPU), a micro processing unit (MPU), a programmable logic device such as a field-programmable gate array (FPGA), a dedicated circuit for executing specific processing such as an application-specific integrated circuit (ASIC), a graphics processing unit (GPU), or a neural processing unit (NPU). Additionally, the type of hardware may be a combination of different types of hardware. In a case where a plurality of pieces of hardware are configured to execute one or a plurality of types of processing of a certain processor, the plurality of pieces of hardware may be present in devices physically separate from each other, or may be present in the same device. In any embodiment, an order of each type of processing performed by the processor is not limited to the above order and may be appropriately changed. In addition, hardware is implemented in a form of an electric circuit (circuitry) in which circuit elements, such as semiconductor elements, are combined.

Further, the program may be software such as firmware or a microcode. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be divided and saved in a plurality of non-transitory computer-readable media present in apparatuses physically separate from each other. The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

The present invention can also be applied to a program and a program product.

As described so far, at least the following matters are described in the present specification.

(1)
An endoscope system comprising:
a light source device including a first light source that outputs first light in a first wavelength range;
an endoscope configured to emit illumination light supplied from the light source device to a subject and image the subject to output an image signal; and
a processor,
wherein the processor is operable in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject, and
the processor is configured to control a light amount of the first light source differently between the observation mode and the treatment mode.

(2)
The endoscope system according to (1),
wherein the light source device includes the second light source, and
the processor is configured to control the light amount of the first light source and a light amount of the second light source.

(3)
The endoscope system according to (2), further comprising:
one or a plurality of illumination windows provided at a tip part of an insertion part of the endoscope,
wherein the first illumination light and the second illumination light are emitted from the one or plurality of illumination windows.

(4)
The endoscope system according to (3),
wherein the processor is configured to turn on the first light source and the second light source in the treatment mode, and the second illumination light includes the first light and the second light.

(5)
The endoscope system according to (4),
wherein a ratio of a light amount of the second light to a light amount of the first light in the second illumination light is larger than a ratio of a light amount of the second light to a light amount of the first light in the first illumination light.

(6)
The endoscope system according to (5),
wherein the processor is configured to output a second image generated from the image signal in the treatment mode.

(7)
The endoscope system according to (6),
wherein the second image is an image in which red brightness is reduced as compared with the first image.

(8)
The endoscope system according to any one of (3) to (7),
wherein the processor is configured to turn on the first light source and the second light source in the observation mode, and
the first illumination light includes the first light and the second light.

(9)
The endoscope system according to (8),
wherein a ratio of a light amount of the first light to a light amount of the second light in the second illumination light is smaller than a ratio of a light amount of the first light to a light amount of the second light in the first illumination light.

(10)
The endoscope system according to any one of (3) to (9),
wherein the light source device includes a third light source that outputs third light in a third wavelength range different from the first wavelength range and the second wavelength range,
peak wavelengths of the second light and the third light are 580 nm or more and 900 nm or less and are different from each other, and
the processor is configured to cause the second light to be emitted and causes the third light not to be emitted in the treatment mode.

(11)
The endoscope system according to any one of (3) to (10),
wherein the processor is configured to output, in the treatment mode, information indicating irradiation energy of the second light in a treatment region to which the second illumination light is emitted based on information on a distance from the tip part of the insertion part of the endoscope to the treatment region and light amount information of the second light.

(12)
The endoscope system according to (11),
wherein the processor is configured to output information indicating integrated irradiation energy of the second light calculated from the irradiation energy of the second light and an emission time of the second illumination light.

(13)
The endoscope system according to (11) or (12),
wherein the processor is configured to calculate the irradiation energy for each of a plurality of the treatment regions having different imaging ranges of the endoscope.

(14)
The endoscope system according to any one of (3) to (13),
wherein the first illumination light and the second illumination light are emitted to a range wider than an imaging range of the endoscope.

(15)
The endoscope system according to any one of (3) to (14),
wherein the processor is configured to repeatedly switch the first illumination light and the second illumination light at an interval defined in advance in the treatment mode and
output a third image generated from the image signal captured during emission of the first illumination light.

(16)
The endoscope system according to any one of (3) to (15),
wherein the processor is configured to control, in the observation mode, luminance of the first light based on luminance information calculated from the image signal, and control, in the treatment mode, luminance of the second light without using the luminance information calculated from the image signal.

(17)
The endoscope system according to (1), further comprising:
an irradiation instrument including the second light source and a long probe that emits the second illumination light including the second light from a tip part,
wherein the first illumination light is emitted from an illumination window provided at the tip part of an insertion part of the endoscope in the observation mode, and
the second illumination light is emitted from the irradiation instrument inserted from a forceps channel of the endoscope in the treatment mode.

(18)
The endoscope system according to (17),
wherein the processor is configured to output information indicating the irradiation energy of the second light from light amount information of the second light that is acquired from the irradiation instrument and information on a distance between the irradiation instrument or the tip part of the insertion part of the endoscope and a treatment region to which the second illumination light is emitted.

(19)
The endoscope system according to any one of (1) to (18),
wherein the processor is configured to output, in the treatment mode, guide information for guiding a position of an insertion part of the endoscope for emitting the second illumination light, by using an image signal acquired from the endoscope.

(20)
The endoscope system according to any one of (1) to (19), further comprising:
a holding mechanism configured to hold a position of a tip part of an insertion part of the endoscope in a lumen of the subject.

(21)
The endoscope system according to any one of (1) to (20),
wherein a peak wavelength of the second light is 580 nm or more and 900 nm or less.

(22)
The endoscope system according to (21),
wherein a peak wavelength of the first light is 400 nm or more and 580 nm or less.

(23)
The endoscope system according to any one of (1) to (22),
wherein the second illumination light is emitted to a duodenum for treatment of diabetes in the treatment mode.

(24)
The endoscope system according to any one of (1) to (23),
wherein the first illumination light is white light.

(25)
The endoscope system according to (10),
wherein the processor is configured to cause only the second light source among the second light source and the third light source to emit light in the observation mode, and cause the second light source and the third light source to emit light in the treatment mode.

(26)
The endoscope system according to (6),
wherein the second image is an image generated with a gain smaller than a gain of the first image.

(27)
A control method of a processor of an endoscope system including a light source device including a first light source that outputs first light in a first wavelength range, an endoscope configured to emit illumination light supplied from the light source device to a subject and image the subject to output an image signal, and the processor, the control method comprising:
operating the processor in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject; and
causing the processor to control a light amount of the first light source differently between the observation mode and the treatment mode.

(28)
A non-transitory computer-readable storage medium storing a control program of a processor of an endoscope system including a light source device including a first light source that outputs first light in a first wavelength range, an endoscope configured to emit illumination light supplied from the light source device to a subject and image the subject to output an image signal, and the processor, the control program causing the processor to:
operate in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject; and
control a light amount of the first light source differently between the observation mode and the treatment mode.

### Explanation of References

2: endoscope system
10: endoscope
11: control device
12: connector
13: insertion part
14, 92a: tip part
15: operation unit
16: treatment tool insertion port
16a: treatment tool channel
17: universal cord
18: endoscope connector
18A: first connector case
18B: second connector case
18C: third connector case
19: monitor
20: light guide rod
21: air supply mouthpiece
22: image signal transmission connector
22A: window
23, 42: illumination window
24: air/water supply connector
25: balloon connector
26: ventilation connector
30: imaging unit
31: power supply generation unit
32: A/D converter
33: DSP
34: image signal modulation unit
35: image signal transmission unit
36: power reception unit
38: CPU
39, 57: signal conversion unit
40, 56: signal transmission and reception unit
40A, 56B: light-emitting device
40B, 56A: light-receiving device
41: light guide
51: power feed unit
52: power supply circuit
53: image signal reception unit
54: image signal demodulation unit
55: signal processing circuit
58: controller
59: light source device
60: optical member
61 to 64, 94 to 97: dichroic mirror
66: condenser lens
70: photodetector element
71: A light detector element
72: G light detector element
73: V light detector element
74: B light detector element
75: R light detector element
80: light source
81: A light source
82: G light source
83: V light source
84: B light source
85: R light source
90: irradiation instrument
91: housing
92: probe
92b: scale
93: optical path
100: power supply
110: endoscopic image
111: guidance display
200: duodenum
211: emission range
212: imaging range
221: observation illumination light
222: treatment illumination light
300: balloon

What is claimed is:

1. An endoscope system comprising:
a light source device including a first light source that outputs first light in a first wavelength range;
an endoscope configured to emit illumination light supplied from the light source device to a subject and image the subject to output an image signal; and
a processor,
wherein the processor is operable in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject, and
the processor is configured to control a light amount of the first light source differently between the observation mode and the treatment mode.

2. The endoscope system according to claim 1,
wherein the light source device includes the second light source, and
the processor is configured to control the light amount of the first light source and a light amount of the second light source.

3. The endoscope system according to claim 2, further comprising:
one or a plurality of illumination windows provided at a tip part of an insertion part of the endoscope,
wherein the first illumination light and the second illumination light are emitted from the one or plurality of illumination windows.

4. The endoscope system according to claim 3,
wherein the processor is configured to turn on the first light source and the second light source in the treatment mode, and
the second illumination light includes the first light and the second light.

5. The endoscope system according to claim 4,
wherein a ratio of a light amount of the second light to a light amount of the first light in the second illumination light is larger than a ratio of a light amount of the second light to a light amount of the first light in the first illumination light.

6. The endoscope system according to claim 5,
wherein the processor is configured to output a second image generated from the image signal in the treatment mode.

7. The endoscope system according to claim 6,
wherein the second image is an image in which red brightness is reduced as compared with the first image.

8. The endoscope system according to claim 3,
wherein the processor is configured to turn on the first light source and the second light source in the observation mode, and
the first illumination light includes the first light and the second light.

9. The endoscope system according to claim 8,
wherein a ratio of a light amount of the first light to a light amount of the second light in the second illumination light is smaller than a ratio of a light amount of the first light to a light amount of the second light in the first illumination light.

10. The endoscope system according to claim 3,
wherein the light source device includes a third light source that outputs third light in a third wavelength range different from the first wavelength range and the second wavelength range,
peak wavelengths of the second light and the third light are 580 nm or more and 900 nm or less and are different from each other, and
the processor is configured to cause the second light to be emitted and cause the third light not to be emitted in the treatment mode.

11. The endoscope system according to claim 3,
wherein the processor is configured to output, in the treatment mode, information indicating irradiation energy of the second light in a treatment region to which the second illumination light is emitted based on information on a distance from the tip part of the insertion part of the endoscope to the treatment region and light amount information of the second light.

12. The endoscope system according to claim 11,
wherein the processor is configured to output information indicating integrated irradiation energy of the second light calculated from the irradiation energy of the second light and an emission time of the second illumination light.

13. The endoscope system according to claim 11,
wherein the processor is configured to calculate the irradiation energy for each of a plurality of the treatment regions having different imaging ranges of the endoscope.

14. The endoscope system according to claim 3,
wherein the first illumination light and the second illumination light are emitted to a range wider than an imaging range of the endoscope.

15. The endoscope system according to claim 3,
wherein the processor is configured to repeatedly switch the first illumination light and the second illumination light at an interval defined in advance in the treatment mode and output a third image generated from the image signal captured during emission of the first illumination light.

16. The endoscope system according to claim 3,
wherein the processor is configured to control, in the observation mode, luminance of the first light based on luminance information calculated from the image signal, and control, in the treatment mode, luminance of the second light without using the luminance information calculated from the image signal.

17. The endoscope system according to claim 1, further comprising:
an irradiation instrument including the second light source and a long probe that emits the second illumination light including the second light from a tip part,
wherein the first illumination light is emitted from an illumination window provided at the tip part of an insertion part of the endoscope in the observation mode, and
the second illumination light is emitted from the irradiation instrument inserted from a forceps channel of the endoscope in the treatment mode.

18. The endoscope system according to claim 17,
wherein the processor is configured to output information indicating the irradiation energy of the second light from light amount information of the second light that is acquired from the irradiation instrument and information on a distance between the irradiation instrument or the tip part of the insertion part of the endoscope and a treatment region to which the second illumination light is emitted.

19. The endoscope system according to claim 1,
wherein the processor is configured to output, in the treatment mode, guide information for guiding a position of an insertion part of the endoscope for emitting the second illumination light, by using an image signal acquired from the endoscope.

20. The endoscope system according to claim 1, further comprising:
a holding mechanism configured to hold a position of a tip part of an insertion part of the endoscope in a lumen of the subject.

21. The endoscope system according to claim 1,
wherein a peak wavelength of the second light is 580 nm or more and 900 nm or less.

22. The endoscope system according to claim 21,
wherein a peak wavelength of the first light is 400 nm or more and 580 nm or less.

23. The endoscope system according to claim 1,
wherein the second illumination light is emitted to a duodenum for treatment of diabetes in the treatment mode.

24. A control method of a processor of an endoscope system including a light source device including a first light source that outputs first light in a first wavelength range, an endoscope configured to emit illumination light supplied from the light source device to a subject and image the subject to output an image signal, and the processor, the control method comprising:
operating the processor in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject; and
causing the processor to control a light amount of the first light source differently between the observation mode and the treatment mode.

25. A non-transitory computer-readable storage medium storing a control program of a processor of an endoscope system including a light source device including a first light source that outputs first light in a first wavelength range, an endoscope configured to emit illumination light supplied from the light source device to a subject and image the subject to output an image signal, and the processor, the control program causing the processor to:
operate in an observation mode in which first illumination light including at least the first light is emitted from the endoscope to the subject and a first image generated from the image signal is output, and in a treatment mode in which second illumination light including second light output from a second light source in a second wavelength range different from the first wavelength range is emitted to the subject; and
control a light amount of the first light source differently between the observation mode and the treatment mode.
